(19) United States
(12) Patent Application Publication
(10) Pub. No.: US 2010/0274295 A1
(43) Pub. Date: Oct. 28, 2010

(54) MEDICAL IMPLANT CONFIGURED TO DELIVER A THERAPEUTIC SUBSTANCE

(75) Inventors: Thomas A. Carls, Memphis, TN (US); Newton H. Metcalf, JR., Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/429,295

(22) Filed: Apr. 24, 2009

(51) Int. Cl. A61B 17/86

(52) U.S. Cl. 606/305; 606/301; 606/302

(57) ABSTRACT

Embodiments of the invention comprise a medical implant that delivers a therapeutic substance. In some embodiments, a component that delivers a therapeutic substance is a cap secured to a portion of a fastener such as a surgical construct, surgical screw, pedicle screw, spinal rod cross-link, or other connection element or device.

### FIELD OF THE INVENTION

The present invention relates generally to the field of medical implants, and more particularly relates to a medical implant that is configured to deliver a therapeutic substance.

### BACKGROUND

The use of therapeutic substances in combination with medical implants is a growing trend and has beneficial characteristics in many treatments. Therapeutic substances may be useful in promoting healing, fighting infection and disease by killing various pathogens such as bacteria, viruses, and microorganisms, promoting favorable cellular activity, killing cancer cells, or any of a wide variety of beneficial results. It may be advantageous to associate a therapeutic substance with a medical implant where the medical implant is implanted in a particularly advantageous location for effective application of the therapeutic substance.

It is a continuing challenge in the art to provide implantable medical devices that may be conveniently and securely placed to deliver effective amounts of therapeutic substances in effective locations. Improved devices may provide secure connection to anatomical structures or to other implant structures. It may be favorable for some improved devices that are capable of delivering a therapeutic substance to securely couple the device with connection points of existing implant structures so that limited or no alteration to existing implants is necessary to implement the implantable medical devices. It may be advantageous to provide implantable medical devices capable of delivering a therapeutic substance that may be placed in a sequence that is complementary to existing surgical procedures.

### SUMMARY

An embodiment of the invention is a medical implant configured to deliver a therapeutic substance. The medical implant embodiment may include a fastener with a connection element. The connection element is configured to transfer a force which may be applied to manipulate at least a part of the fastener. The medical implant embodiment may also include a cap with a body that contains the therapeutic substance, and a linking mechanism configured to couple at least with the connection element to secure the cap to the fastener. The body may be configured to release at least a portion of the therapeutic substance when the body is exposed to an at least in part aqueous substance.

Another embodiment of the invention is a cap configured to couple with a fastener and configured to deliver a therapeutic substance. The cap may include a body that contains the therapeutic substance, and a linking mechanism configured to couple at least with a connection element of the fastener to secure the cap to the fastener. The cap may also capture at least a portion of the fastener between at least two opposing sides of the cap. The body may also be configured to release at least a portion of the therapeutic substance when the body is exposed to an at least in part aqueous substance.

Yet another embodiment of the invention is a cap in combination with a break-off fastener. The cap may be configured to deliver a therapeutic substance. The cap embodiment may include a body that contains the therapeutic substance and a linking mechanism couple at least with the break-off fastener to secure the cap to the break-off fastener. The cap of some embodiments is disposed at least in part in an internal opening in the break-off fastener. At least a portion of the cap is disposed in a part of the break-off fastener that remains in the medical implant after the break-off fastener is operated and broken off in some embodiments. The body may be configured to release at least a portion of the therapeutic substance when the body is exposed to an at least in part aqueous substance.

### BRIEF DESCRIPTION OF THE DRAWINGS

FIG. 1 is an elevation view of embodiments of a medical implant in use in lumbar and sacral spinal segments.

FIG. 2 is a perspective view of a prior art pedicle screw.

FIG. 3 is a perspective view of an embodiment of a medical implant.

FIG. 4 is a perspective view of a portion of an embodiment of the medical implant of FIG. 3 with part of the medical implant rotated to see interior components.

FIG. 5 is a plan view of an embodiment of the medical implant of FIG. 4.

FIG. 6 is an elevation view of an embodiment of a medical implant with portions of the medical implant shown in cross-section.

FIG. 7 is an elevation view of an embodiment of a medical implant with portions of the medical implant shown in cross-section.

FIG. 8 is a perspective view of an embodiment of a medical implant.

FIG. 9 is a perspective view of an embodiment of a medical implant.

FIG. 10 is a perspective view of a portion of an embodiment of the medical implant of FIG. 9 with part of the medical implant rotated to see interior components.

FIG. 11 is a perspective view of an embodiment of a medical implant with a portion of the implant shown cut away to illustrate additional components.

FIG. 12 is a bottom plan view of a portion of an embodiment of the medical implant of FIG. 11.

FIG. 13 is a perspective view of an embodiment of a medical implant.

### DETAILED DESCRIPTION

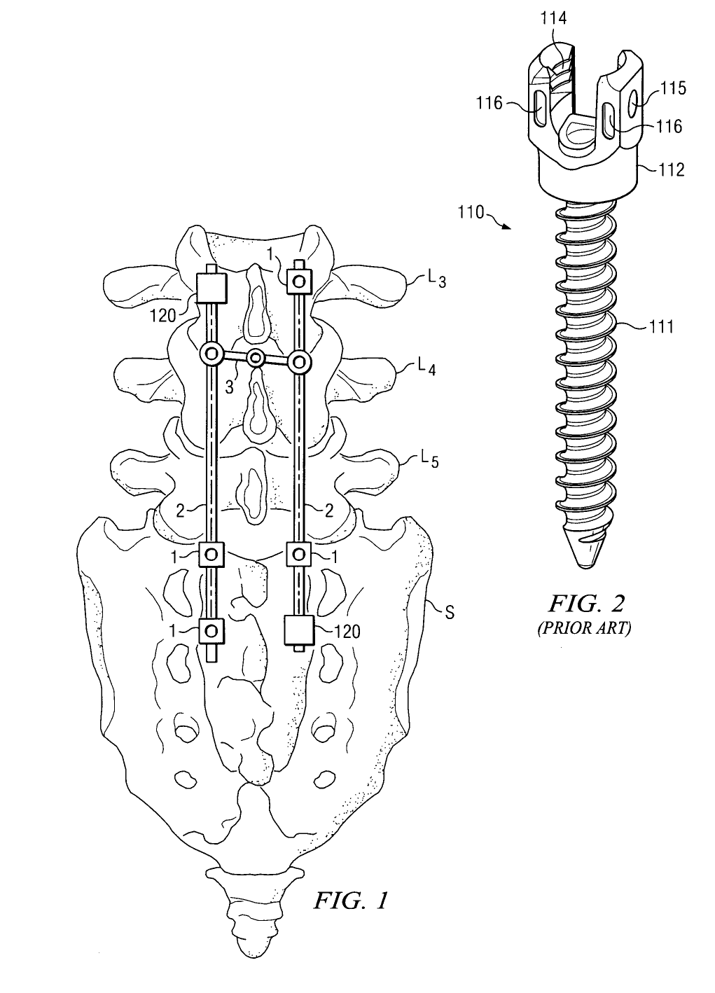

A rod and screw based spinal construct implanted in lumbar and sacral regions of a human spine is illustrated in FIG. 1. A sacrum S is shown, to which lumbar vertebrae L₅, L₄, and L₃, are consecutively connected. Pedicle screws 1 are shown connected to the sacrum S, and the L₃ and L₄ vertebrae. Two spinal rods 2 are coupled between several of the pedicle screws 1 to stabilize the illustrated spinal segment. A cross-link 3 is also shown connecting two spinal rods 2. A cap 120 of a medical implant 100 is shown covering portions of the spinal construct in two places in the illustrated embodiment.

A prior art pedicle screw or fastener 110 is illustrated in FIG. 2. The fastener 110 shown is a multi-axial pedicle screw such as the CD HORIZON® LEGACY™ pedicle screw offered by Medtronic, Inc. The fastener 110 includes a shank 111 and a receiver 112. A spinal rod may be placed in the receiver 112. The receiver 112 is also illustrated with threads 114 that are configured to engage with a set screw. An example set screw 313 engaged with a receiver 312 is illustrated in FIG. 7. Several openings 115, 116 in the receiver 112 are shown in FIG. 2. Openings of various embodiments may be of any shape or configuration and intended for any purpose. The opening 115 that is shown may be, for example and without limitation, useful for grasping the receiver 112 with an instrument used to manipulate the position of the receiver 112 relative to other pedicle screws or implant components, or relative to the anatomy of a patient. The openings 116 may be similarly employed and may particularly be useful for grasping by a rod reduction instrument that pushes a spinal rod down into the receiver 112.

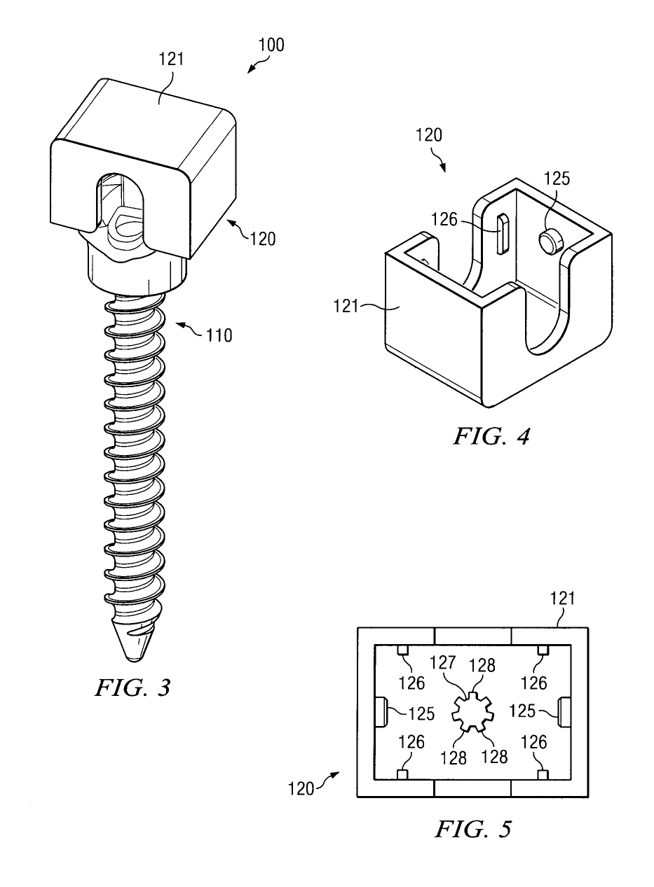

A medical implant 100 configured to deliver a therapeutic substance is illustrated in FIGS. 3-5. The medical implant 100 may include a fastener 110 and a cap 120. The illustrated fastener 110 is a pedicle screw, but in other embodiments of the fastener may be any variety of fasteners. For example and without limitation, the fastener may be a part of a cross-link device as is illustrated in FIGS. 1, 11, and 13, an orthopedic bone screw for use alone or in combination with a plate, an interbody implant, or other device, a nut such as an external hex nut, or a component with an internal mating surface or volume such as an opening, an internal hex, an internal star, an internal thread, or any surface or volume to which a connection may be made. Any of these structures may also be described as a connection element of the fastener 110 or other devices in various embodiments. A connection element may be configured to transfer a force which may be applied to manipulate at least a part of the fastener 110. Such a force may be a turning force or other torque, a pulling force, or any force effective to manipulate at least a part of a fastener. In the illustrated embodiment, the openings 115, 116 (FIG. 2) serve as connection elements for the fastener 110. The connection element comprising opening 115 that is on an exterior side of the receiver member 112 may also include an opening on an opposite exterior side (not shown) of the receiver member 112. Similarly, the connection element comprising one or both of the openings 116 that are on an exterior side of the receiver member 112 may also include one or more openings (not shown) on an opposite exterior side of the receiver member 112.

The cap 120, as illustrated in FIGS. 1 and 3-5 includes a body 121 that contains a therapeutic substance, and a linking mechanism configured to couple at least with the connection element of the fastener 110 to secure the cap 120 to the fastener 110. In the illustrated embodiment, the linking mechanism includes one or more of protrusions 125, 126. As shown in FIGS. 4 and 5, the cap 120 may include pairs of protrusions 125, 126 on opposite sides of the cap 120. When placed over a fastener, as illustrated with the fastener 110 in FIG. 3, the cap 120 captures portions of the fastener 110 between opposing sides of the cap 120. As shown, the cap 120 captures portions of the fastener 110 between two sets of opposing sides of the cap 120. In other embodiments, a cap may only capture a fastener between one set of opposing sides, or may couple with the fastener in another manner.

In some embodiments, the linking mechanism may also include an insert 127 as illustrated in FIG. 5. The insert 127 is also a protrusion under the terms herein, as noted with regard to protrusions 125, 126 above. As shown in FIG. 5, the illustrated insert 127 includes resilient members, such as the fingers 128, which may interact with a connection element of a fastener to couple the cap 120 to the fastener 110. The fingers 128 may comprise a flexible material that may be compressed within an internal opening in a fastener. Compressed fingers 128 may create greater force against a connection element of a fastener and make the coupling between a cap and a fastener more secure. The fingers 128 may alternatively or in addition be flexible at their bases or along their lengths so that the fingers 128 bend when being pushed into a fastener and then extend to resist disengagement of the cap 120 from the fastener 110 when subject to a separating bias. By way of non-limiting example, the fastener 110 of FIG. 5 may include a connection element that is a set screw, similar to the set screw 313 shown in FIG. 7, and the insert 127 linking mechanism may couple with an opening for a tool in a set screw that is engaged with the threads 114 of the receiver 112 to secure the cap 120 to the fastener 110. The insert 127 may also be shaped in approximately the same shape as but slightly larger than an opening in the fastener 110 in which the insert 127 is placed to compress the insert 127 and produce a tight fit between the fastener 110 and the cap 120.

In the illustrated embodiment, the body 121 is configured to release at least a portion of the therapeutic substance when the body 121 is exposed to an at least in part aqueous substance. The aqueous substances of some embodiments are bodily fluids. The bodily fluids may contact all or a part of the body 121 when the body 121 is implanted and closed at least in part within a patient. In some embodiments, linking mechanisms of the cap 120 may also contain and be configured to release therapeutic substance.

Embodiments of the cap 120 in whole or in part may be constructed of biocompatible materials of various types. Examples of cap materials include, but are not limited to, non-reinforced polymers, reinforced polymer composites, metals, ceramics and combinations thereof. In some embodiments, the cap 120 may be constructed of sections of bone or other tissues. Tissue materials include, but are not limited to, autograft, allograft, or xenograft, and may be resorbable or non-resorbable in nature. Examples of other tissue materials include hard tissues, connective tissues, demineralized bone matrix, and combinations thereof.

All or a part of the cap 120 may include a polymeric body configured to elute the therapeutic substance. The polymeric body may further elute the therapeutic substance at a predetermined rate. Alternatively or in addition, the cap 120 may at least in part be porous, and the therapeutic substance may be at least in part disposed in the pores of the cap 120.

The cap 120 in whole or in part may comprise a polymeric material into or onto which a therapeutic substance is incorporated. Any polymeric material may be used. The polymeric material may be biocompatible and capable of presenting or eluting the therapeutic substance in an effective amount. Biocompatible polymers may be obtained from natural or synthetic sources, and may be bioresorbable. Examples of natural materials of which the polymer may be composed include collagen, elastin, silk, and demineralized bone matrix. Other examples of suitable polymeric materials include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; polytetrafluoroethylene (PTFE); polyethylene, low density polyethylene; polymethylmethacrylate (PMMA); polyetheretherketone (PEEK); and polyetherketoneketone (PEKK). The polymer may also be a polymeric hydroxyethylmethacrylate (PHEMA). Suitable bioresorbable synthetic polymers include poly(L-lactide), poly(D,L-lactide), poly(L-co-D,L-lactide), polyglycolide, poly(lactide-co-glycolide), poly(hydroxybutyrate), poly(hydroxyvalerate), tyrosine-derived polycarbonate, polyanhydride, polyorthoester, polyphosphazene, poly(dioxanone), and polyglyconate. Other similar polymers known to the art may be used and various mixtures of polymers may be combined to adjust the properties of the composition as desired.

A therapeutic substance may be incorporated into or coated on a polymeric material of the cap 120 using any known or developed technique. For example, the therapeutic substance may be adhered to a surface of any part of the cap 120, adsorbed into the cap 120, or compounded into the polymeric material that forms the cap 120. Accordingly, the therapeutic substance may be embedded, coated, mixed or dispersed on or in the material of the cap 120. A coating method may be determined by the material of the cap and the therapeutic substance utilized. Such methods include but are not limited to, dipping, spraying, rolling, plating and embedding the coating into the surface by any means. For example, a polymeric cap may be coated by dip or spray coating polymeric resin and crosslinker with the therapeutic substance as substituent or dissolved within the polymer. Curing may be achieved chemically, photochemically or thermally. Other common methods include dip or spray coating water insoluble resin containing a therapeutic substance followed by drying or grafting directly onto the substrate chemically or photochemically.

Additional examples of ways to form at least a portion of the cap 120 include blending a therapeutic substance with a polymer and then forming the polymer into the cap 120, or portion of the cap 120. Alternatively, the therapeutic substance may be in a solution with the polymer to form a coating. The therapeutic substance may be attached to a polymeric material by a chemical modification of the surface such as surface grafting by hydrolyzable linkage of the therapeutic substance to the surface or by photolinking the therapeutic substance to the surface. Surface polymerization, derivatization or absorption may also be used. Other examples of obtaining a surface bound therapeutic substance include any existing means, such as ion implantation, chemical modification of the surface, photochemical or chemical grafting or formation of a crosslinked surface immobilized network. Silver ions, where used, may be deposited on the surface of the cap 120 by vacuum deposition, ion sputtering or surface deposition, among others. The surface of the cap 120 may be pretreated according to known methods such as plasma treatment prior to exposure to the coating material. Where solvents are present in the therapeutic substance, such solvents may be biocompatible if residue remains after the therapeutic substance is applied.

Metals which can be used to form all or a part of the cap 120 include but are not limited to stainless steel and other steel alloys, cobalt chrome alloys, tantalum, titanium, titanium alloys, titanium-nickel alloys such as Nitinol and other superelastic or shape-memory metal alloys. Metals can be formed into supportive frameworks by a variety of manufacturing procedures including combustion synthesis, plating onto a “foam” substrate, chemical vapor deposition (see U.S. Pat. No. 5,282,861), lost mold techniques (see U.S. Pat. No. 3,616,841), foaming molten metal (see U.S. Pat. Nos. 5,281,251, 3,816,952 and 3,790,365), and replication of reticulated polymeric foams with a slurry of metal powder. Sintering of metals and polymers of various types and other methods of forming porous structures to make all or part of the cap 120 may be accomplished as disclosed at least in U.S. Pat. Nos. 6,572,619, and 6,673,075. Metal particles may have to be fused at elevated temperatures and therefore cannot be readily formed directly on surfaces which would be adversely affected by the fusion temperature needed for metal particles. Metal particles may be bonded onto a surface with an adhesive acting to bond the particles with a particle-surface coating matrix which does not fill the pores. By proper selection of the amount (the relative amount of polymer binder to metal), the pore size can be tightly controlled and the metal/binder materials applied to a wide array of surfaces. Various types of polymer binders such as thermoplastic binders (applied by melting the polymer of applied from solution, dispersion, emulsion or suspension or even direct polymerization on the surface of the polymers by heat, catalysis, or radiation), thermoset binders (also provided by reaction on the surface of the particles) or by fusion of the particles (with or without additional cross linking), or the like, may be used. Among the useful classes of polymers would be at least polyamides, polyacrylates, polyurethanes, silicon polymers (e.g., polysiloxanes, silicone rubbers, siloxane graft or block polymers or copolymers, etc.), polyester resins, highly fluorinated resins (e.g., polytetrafluoroethylene), polyimides, and the like. These same classes of polymers may also comprise the mass of the therapeutic substance delivery element itself. Particularly when latices are used to mold the cap 120 or particles are fused (thermally or by solvents) to form the cap 120, the degree of pressure applied, the level of heat applied, the duration of the solvent, and other obvious parameters may be used to control the degree of fusion of the polymer and its degree of porosity. Porosity can also be created in polymeric materials useful for the cap 120 by including a soluble or leachable or flowable pore-leaving component with the polymer, forming the cap, and then removing the pore-leaving component. Techniques in this category include mixing a highly soluble particle (soluble in a solvent in which the polymer is not soluble), such as NaCl, into the polymer. Casting or molding the cap 120, and then leaching out or dissolving out the salt with water. By controlling the volume of salt, and the size of the salt particles, the pore size can be readily controlled. Alternatively, it is known to mix a non-solvent liquid from the polymer to form an emulsion or dispersion. When the polymer is solidified as the cap 120 or component of the cap 120, the non-solvent remains as a dispersed phase which can be readily removed from the cap by washing. Thermoplastic particles may be fused under controlled pressure to form the cap 120 with controlled pore size, as with the ceramics and the metal particles.

Ceramic materials that can be used to form all or a part of the cap 120 include but are not limited to inorganic metal oxides such as aluminum oxide, silica, zirconium oxide, titanium oxide, and composites of mixtures of inorganic oxides. Ceramic materials can be fabricated at both room temperatures and elevated temperatures and so can be provided as both separate caps or as caps on substrates which could suffer from exposure to elevated temperatures. For example, many ceramics can be formed by solidification (dehydration) of sol-gel dispersions or suspensions of inorganic oxide particles. Other ceramics must be dehydrated and bonded together at elevated temperatures. By controlling the pressure applied to the ceramic material during hardening or fusing, the pore size can be controlled. The use of ceramic-forming particles of different average sizes will also affect the average pore size according to conventional packing and distribution laws. The structure of the cap 120 may be altered to control the elution rate or release rate of the drug. For example, the size of the pores on the outer surface which are exposed to the body liquids is a significant rate limiting factor in the design, while at the same time, the pore size controls the amount of therapeutic substance that can be retained within the cap 120. As the pore size increases internally, larger amounts of therapeutic substance may be stored, while larger pore sizes on the surface increase the therapeutic substance release rate. One design would therefore have pore openings on the surface of the cap 120 with smaller average diameters of the pores than larger pores within the body of the cap 120 which are fluid transferring connected to the pores on the surface of the cap 120. In some embodiments, the interior pores have average pore dimensions which are at least 10-50% greater in average diameter than the pores open at the surface of the cap 120. Combinations of the materials noted above for use in making the cap 120 or portions of the cap 120 may be used in any effective amount or assembly.

The therapeutic substance may comprise one or more of the following: antibiotics, antiseptics, analgesics, bone growth promoting substances, anti-inflammatants, anti-arrhythmics, anti-coagulants, antifungal agents, steroids, enzymes, immunosuppressants, antithrombogenic compositions, vaccines, hormones, growth inhibitors, growth stimulators, and the like. The therapeutic substance may be any drug or bioactive agent which can serve a useful therapeutic or even diagnostic function when released into a patient. More than one therapeutic substance may be present in or on the cap for a particular treatment within the scope of the invention.

Any antibiotic suitable for use in a human may be used in accordance with various embodiments of the invention. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other antibiotics may also be used.

It may be desirable that the one or more antibiotics selected kill or inhibit the growth of one or more bacteria that are associated with infection following surgical implantation of a medical device. Such bacteria may include Stapholcoccus aureus and Staphlococcus epidermis. The one or more antibiotics selected may be effective against strains of bacteria that are resistant to one or more antibiotics. To enhance the likelihood that bacteria will be killed or inhibited, it may be desirable to combine one or more antibiotics. It may also be desirable to combine one or more antibiotics with one or more antiseptics. Agents having different mechanisms of action and/or different spectrums of action may be most effective in achieving such an effect. In a particular embodiment, a combination of rifampin and minocycline is used.

Any antiseptic suitable for use in a human may be used as or as part of the therapeutic substance. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver sulfadiazine and alcohols. It may be desirable that the one or more antiseptics selected kill or inhibit the growth of one or more microbes that are associated with infection following surgical implantation of a medical device. Such bacteria may include Stapholcoccus aureus, Staphlococcus epidermis, Pseudomonus auruginosa, and Candidia. To enhance the likelihood that microbes will be killed or inhibited, it may be desirable to combine one or more antiseptics. It may also be desirable to combine one or more antiseptics with one or more antibiotics. Antimicrobial agents having different mechanisms of action and/or different spectrums of action may be most effective in achieving such an effect. In a particular embodiment, a combination of chlorohexidine and silver sulfadiazine is used.

A therapeutic substance, such as an antibiotic or antiseptic, may be present in the cap 120 at any concentration effective, either alone or in combination with another therapeutic substance, to prevent or treat an infection. Generally, a therapeutic substance may be present in the cap 120 at a range of between about 0.5% and about 20% by weight. For example, the therapeutic substance may be present in the cap 120 at a range of between about 0.5% and about 15% by weight or between about 0.5% and about 10% by weight.

The therapeutic substance may comprise an antimicrobial material including metals known to have antimicrobial properties such as silver, gold, platinum, palladium, iridium, tin, copper, antimony, bismuth, selenium and zinc. Compounds of these metals, alloys containing one or more of these metals, or salts of these metals may be coated onto the surface of the cap 120 or added to the material from which the cap 120 is made during the manufacture of the cap 120 or compounded into the base material. One therapeutic substance will contain silver ions and may be obtained through the use of silver salts, such as silver acetate, silver benzoate, silver carbonate, silver iodate, silver iodide, silver lactate, silver laurate, silver nitrate, silver oxide, silver palmitate, silver protein, or silver sulfadiazine, among others. In an embodiment where selenium is used, the selenium may be bonded to the surface of the cap 120, providing an antimicrobial coating.

Therapeutic substances may be chosen based upon a particular application anticipated for a cap. For example, it may be desirable to use a timed release or leachable content material for a particular use. The material comprising the cap may also affect the choice of therapeutic substance. For example, metal caps which are to be provided with therapeutic substance coatings may require therapeutic substances which can be coated onto the metal with satisfactory adhesion to resist the harboring of infectious organisms, or the ability to kill such organisms present throughout the use of the cap. Alternatively, where the therapeutic substance is to be compounded into a metal cap prior to its formation into a cap, the therapeutic substance should be selected so that the therapeutic substance can be readily incorporated into the metal of the cap. In some embodiments, the cap 120 may be at least in part titanium and the therapeutic substance will be silver ion.

Likewise, where the cap 120 comprises a polymeric material, the therapeutic substance may be selected such that the therapeutic substance can be used as a coating material. For example, materials such as silver ions, selenium, and silver zeolite may be used. Separately or in addition, any commercially available additives, such as Heathshield®, among others, may be used.

Use of the cap 120 may provide a positive therapeutic effect by a variety of mechanisms, including preventing adherence of an organism to a surface of the cap 120 or adjacent implant structures, providing slow release of a therapeutic substance into the surrounding area, or fixing a source for the therapeutic substance on implant structures for long term effects. The rate of release from a cap, such as the cap 120, is intended to be highly tailored to the specific use of the associated medical device.

The therapeutic substance may also comprise an osteoconductive, osteogenic, or osteoinductive material. For example and without limitation, the therapeutic substance may include various bioceramic materials, calcium phosphate and other members of the calcium phosphate family, fluorapatite, bioactive glass, and collagen-based materials. Members of the calcium phosphate family include materials such as hydroxyapatite, α-tricalcium phosphate, β-tricalcium phosphate, tetracalcium phosphate, dicalcium phosphate dihydrate, octacalcium phosphate, and the like. The therapeutic substance may include an osteoinductive or osteogenic materials such as osteoblast cells, platelet-derived growth factors (PDGFs), bone morphogenetic proteins (BMPs), insulin-like growth factors (IGFs), basic fibroblast growth factor (bFGF), cartilage derived morphogenetic protein (CDMP), growth and differentiation factors (GDFs), LIM mineralization proteins, transforming growth factor beta family (TGF-β), and other bone proteins, such as CD-RAP. These proteins can be recombinantly produced or obtained and purified from an animal that makes the proteins without the use of recombinant DNA technology. Recombinant human BMP is referred to as “rhBMP”; recombinant human GDF is referred to as “rhGDF”. Any bone morphogenetic protein is contemplated, including bone morphogenetic proteins designated as BMP-1 through BMP-18. Mimetics of growth factors can also be used in the devices of the present invention for inducing the growth of bone.

Each BMP may be homodimeric, or may be heterodimeric with other BMPs (e.g., a heterodimer composed of one monomer each of BMP-2 and BMP-6) or with other members of the TGF-β superfamily, such as activins, inhibins and TGF-β 1 (e.g., a heterodimer composed of one monomer each of a BMP and a related member of the TGF-α superfamily). Any of these substances may be used individually or in mixtures of two or more. One or more statins may also be included in the therapeutic substance. Non-limiting examples of statins that may be included in the devices of the present invention include atorvastatin, cerivastatin, fluvastatin, lovastatin, mavastatin, pitavastatin, pravastatin, rosuvastatin and simvastatin. The therapeutic substance may include various other organic species known to induce bone formation, and combinations thereof.

The therapeutic substance may also or in addition include pharmaceuticals that target particular cells, such as but not limited to, cancer cells.

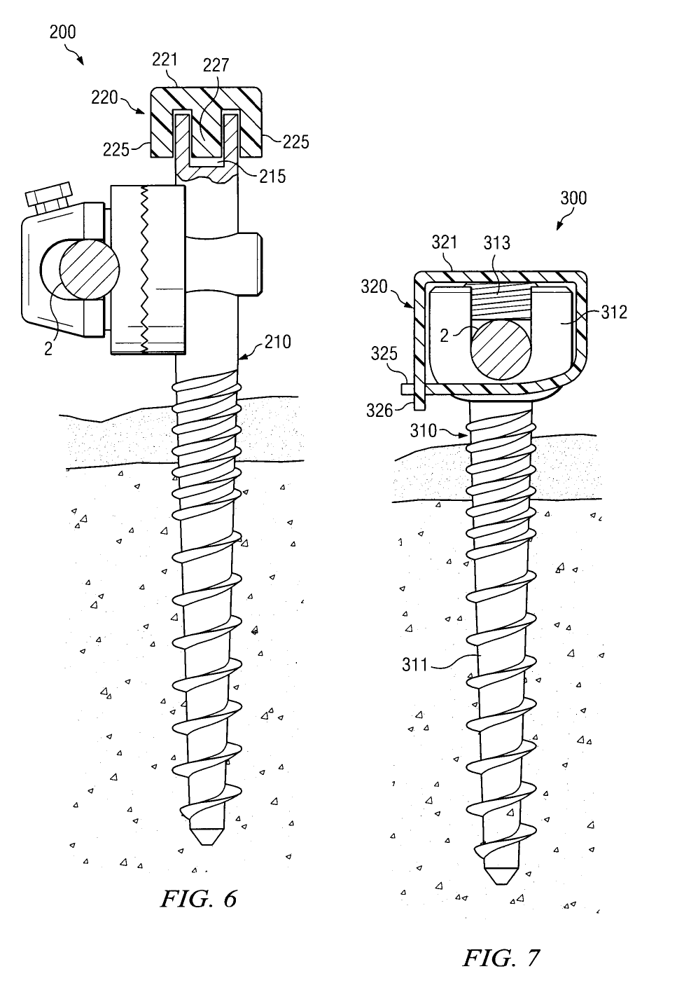

An embodiment of a medical implant 200 configured to deliver a therapeutic substance is illustrated in FIG. 6. The medical implant 200 includes a fastener 210 and a cap 220. In the illustration of FIG. 6, the medical implant 200 is generally viewed in elevation; however, cross-sectional views through a proximal end of the fastener 210, through the cap 220, and through a connected spinal rod 2 are depicted. The illustrated fastener 210 is a pedicle screw. A connection element may be configured to transfer a force which may be applied to manipulate at least a part of the fastener 210. In the illustrated embodiment, the opening 215 in a proximal end of the fastener 210 serves as a connection element for the fastener 210.

The cap 220, as illustrated in FIG. 6 includes a body 221 that contains a therapeutic substance, and a linking mechanism configured to couple at least with the connection element of the fastener 210 to secure the cap 220 to the fastener 210. In the illustrated embodiment, the linking mechanism includes an insert 227 and walls 225. The insert 227 and the walls 225 are protrusions under the terms herein. When placed over a fastener, as illustrated with the fastener 210, the cap 220 captures portions of the fastener 210 between opposing sides, or walls 225 of the cap 220. When in place on the fastener 210, the insert 227 fits into the opening 215 in the fastener 210. The insert 227 of some embodiments includes sizing, fingers, and resilient material similar to the devices described in association with the insert 127 above.

In the illustrated embodiment, the body 221 is configured to release at least a portion of the therapeutic substance when the body 221 is exposed to an at least in part aqueous substance. The aqueous substances of some embodiments are bodily fluids. The materials from which the cap 220 and its component parts, including associated therapeutic substances, are made are essentially the same as the materials from which the cap 120 and its component parts are made, and the description above associated with the cap 120 is applicable to the cap 220.

An embodiment of a medical implant 300 configured to deliver a therapeutic substance is illustrated in FIG. 7. The medical implant 300 includes a fastener 310 and a cap 320. In the illustration of FIG. 7, the medical implant 300 is generally viewed in elevation; however, cross-sectional views through the cap 320 near the fastener 310 and through a connected spinal rod 2 are depicted. The illustrated fastener 310 is a pedicle screw with a shank 311 and a receiver 312. The fastener 310 also includes a set screw 313 that holds the spinal rod 2 in place in the receiver 312 of the fastener 310.

The cap 320, as illustrated in FIG. 7, includes a body 321 that contains a therapeutic substance, and a linking mechanism configured to couple at least with a connection element of the fastener 310 to secure the cap 320 to the fastener 310. In the illustrated embodiment, the linking mechanism includes a first leg 325 and a second leg 326 that wrap around the spinal rod 2 that passes through the fastener 310. The linking mechanism of the illustrated cap 320 couples with the fastener 310 by capturing at least one connection element of the fastener 310. For example, the fastener 310 may include openings in exterior sides of the fastener 310 similar to openings 115, 116, and 215 described above, or a tool opening in the set screw 313. The cap 320 may additionally include an insert (not shown) configured to be inserted into a tool opening in the set screw 313, similar to inserts 127, 227 described above. The first and second legs 325, 326 of the illustrated embodiment connect with one another and wrap around the spinal rod 2 on at least one side of the receiver 312. In some embodiments, both of the first and second legs 325, 326 may connect to each other on both sides of the receiver 312. For example and without limitation, one of the first and second legs 325, 326 may include a strap and the other may include a pawl that engages with the strap such that as the strap is advanced through the pawl, a loop including the first and second legs 325, 326 is reduced in size and the cap 320 is secured. The strap may include ratchet teeth that engage with the pawl to progressively lock the first and second leg 325, 326 together as the strap is advanced through the pawl. In some embodiments, each of the first and second legs 325, 326 may wrap around the spinal rod 2 on one or both sides of the receiver 312, and may or may not connect with one another. When placed over a fastener, as illustrated with the fastener 310, the cap 320 captures portions of the fastener 310 between opposing sides.

In the illustrated embodiment, the body 321 is configured to release at least a portion of the therapeutic substance when the body 321 is exposed to an at least in part aqueous substance. The aqueous substances of some embodiments are bodily fluids. The materials from which the cap 320 and its component parts, including associated therapeutic substances, are made are essentially the same as the materials from which the cap 120 and its component parts are made, and the description above associated with the cap 120 is applicable to the cap 320.

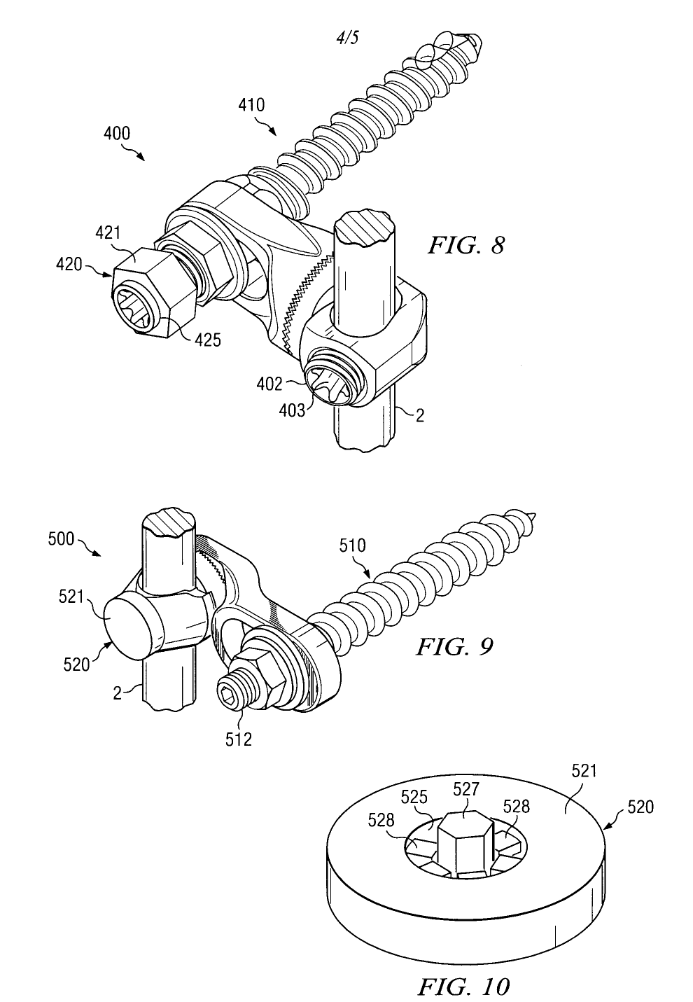

FIGS. 8 and 9 illustrate two similar medical implants 400, 500 that include spinal rod and screw systems with multiple fasteners. The system of FIG. 8 includes fasteners embodied in a fastener 410 and a rod set screw 402. The system of FIG. 9 includes fasteners embodied in a fastener 510 and a rod set screw (not shown). The systems of both FIGS. 8 and 9 respectively couple with spinal rods 2 through similar variable angle coupling mechanisms. An externally threaded proximal end 512 of the fastener 510 is shown in FIG. 9. The proximal end of the fastener 410 includes a similar externally threaded proximal end, but a fully illustrative view of the proximal end of the fastener 410 is obscured by a cap 420. The rod set screw 402 is shown in FIG. 8. A similar rod set screw exists for the spinal rod and screw system of FIG. 9, but the view of the rod set screw is obscured by a cap 520.

The medical implant 400 illustrated in FIG. 8 is configured to deliver a therapeutic substance. The medical implant 400 includes the fastener 410 and the cap 420. The illustrated fastener 410 is a pedicle screw. A connection element may be configured to transfer a force which may be applied to manipulate at least a part of the fastener 410. In the illustrated embodiment, at least the external threads on the proximal end of the fastener 410 serve as a connection element for the fastener 410.

The cap 420, as illustrated in FIG. 8, includes a body 421 that contains a therapeutic substance, and a linking mechanism configured to couple at least with the connection element of the fastener 410 to secure the cap 420 to the fastener 410. In the illustrated embodiment, the linking mechanism includes a hole 425 through the cap 420 with threads on an interior surface of the hole 425 that are configured to mate with the external threads on the proximal end of the fastener 410. The illustrated cap 420 is formed in the shape of a hex nut and may be applied as a nut would be applied. In other embodiments, the cap 420 may be of any operable shape and the hole 425 may or may not extend completely through the cap 420.

In the illustrated embodiment, the body 421 is configured to release at least a portion of the therapeutic substance when the body 421 is exposed to an at least in part aqueous substance. The aqueous substances of some embodiments are bodily fluids. The materials from which the cap 420 and its component parts, including associated therapeutic substances, are made are essentially the same as the materials from which the cap 120 and its component parts are made, and the description above associated with the cap 120 is applicable to the cap 420.

The medical implant 500 illustrated in FIG. 9 is configured to deliver a therapeutic substance. The medical implant 500 includes the rod set screw (not shown) and the cap 520. The rod set screw, as noted above, is an embodiment of a fastener under the terms herein. A connection element may be configured to transfer a force which may be applied to manipulate at least a part of the rod set screw. In the embodiment of FIG. 9, at least a tool opening in the rod set screw (similar to the tool opening 403 in the rod set screw 402 shown in FIG. 8) serves as a connection element for the rod set screw of FIG. 9. Note that the tool opening 403 in the rod set screw 402 is shaped to receive a star pattern tool, and while any functional shape is within embodiments of the device, the tool opening in the rod set screw of FIG. 9 is shaped as an internal hex.

The cap 520, as illustrated in FIGS. 9 and 10, includes a body 521 that contains a therapeutic substance, and a linking mechanism configured to couple at least with the connection element of the rod set screw of FIG. 9 to secure the cap 520 to the rod set screw. In the illustrated embodiment, the linking mechanism includes an insert 527 and walls 525. The insert 527 and the walls 525 are protrusions under the terms herein. When placed over a fastener, such as the rod set screw of FIG. 9, the cap 520 captures portions of the rod set screw between opposing sides, or walls 525 of the cap 520. When in place on the rod set screw, the insert 527 fits into the tool opening in the rod set screw. The insert 527 of some embodiments includes sizing, fingers, and resilient material similar to the devices described in association with the insert 127 above. Additionally or alternatively, the walls 525 may include devices for creating a more secure fit between the cap 520 and the rod set screw such as fingers 528. The fingers 528 may have similar characteristics to the fingers 128 and resilient material of the insert 127 described above.

In the illustrated embodiment, the body 521 is configured to release at least a portion of the therapeutic substance when the body 521 is exposed to an at least in part aqueous substance. The aqueous substances of some embodiments are bodily fluids. The materials from which the cap 520 and its component parts, including associated therapeutic substances, are made are essentially the same as the materials from which the cap 120 and its component parts are made, and the description above associated with the cap 120 is applicable to the cap 520.

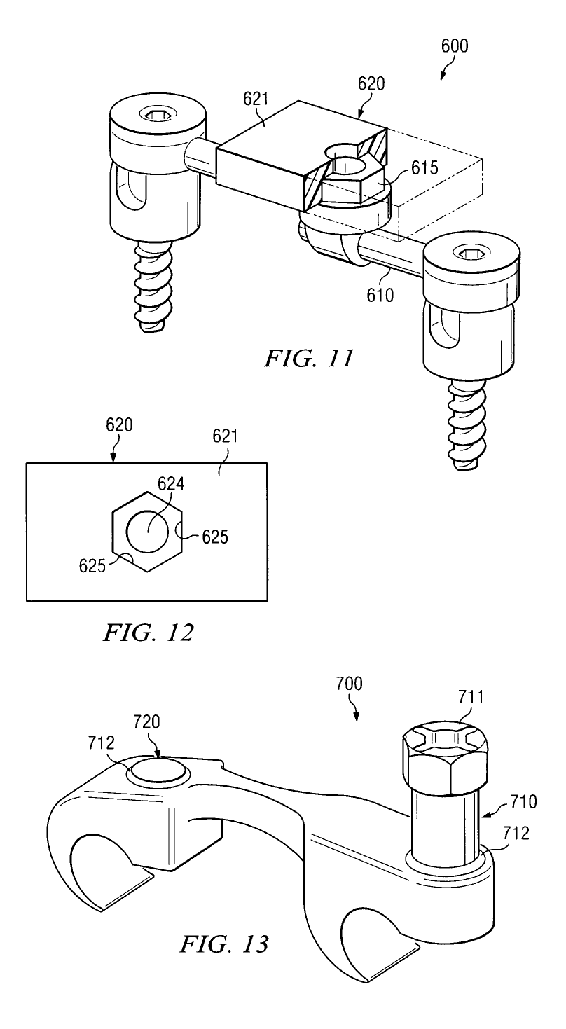

A medical implant 600 illustrated in FIGS. 11 and 12 is configured to deliver a therapeutic substance. The medical implant 600 includes a cross-link 610 and a cap 620. At least portions of the cross-link 610 are an embodiment of a fastener under the terms herein. The cross-link 610 is a part of a larger rod and screw construct in some embodiments. The medical implant 600 under embodiments of the device includes the entire rod and screw construct. A connection element of the cross-link 610 may be configured to transfer a force which may be applied to manipulate at least a part of the cross-link 610. In the embodiment of FIG. 11, at least a nut 615 with an external surface serves as a connection element for the cross-link 610. The nut 615 illustrated is an external hex shape, but embodiments of the device may include any functional shape or surface.

The cap 620, as illustrated in FIGS. 11 and 12, includes a body 621 that contains a therapeutic substance, and a linking mechanism configured to couple at least with the connection element of the cross-link 610 to secure the cap 620 to the cross-link 610. In the illustrated embodiment, the linking mechanism includes a hole in the shape of a hex with walls 625. When placed over a fastener, such as the nut 615, the cap 620 captures portions of the nut 615 between opposing sides, or walls 625 of the cap 620. Additionally or alternatively, the walls 625 may include devices for creating a more secure fit between the cap 620 and the cross-link 610. In some embodiments, the cap 620 may be secured to the cross-link 610 by applying an adhesive between the cap 620 and the cross-link 610. Use of an adhesive between any cap or fastener of any embodiment of the devices disclosed herein may be employed. The general shape of the cap 620 is rectangular, with a longer dimension of the rectangle oriented along the cross-link 610 as illustrated. However, the shape of cap 620 may be of any functional shape. In some embodiments, the cap 620 may be shaped to particularly fit between spinal structures, such as spinal processes. The cap 620 of some embodiments may include an access hole 624 so that components of the cross-link 610 may be accessed after the cap 620 is coupled to the cross-link 610.

In the illustrated embodiment, the body 621 is configured to release at least a portion of the therapeutic substance when the body 621 is exposed to an at least in part aqueous substance. The aqueous substances of some embodiments are bodily fluids. The materials from which the cap 620 and its component parts, including associated therapeutic substances, are made are essentially the same as the materials from which the cap 120 and its component parts are made, and the description above associated with the cap 120 is applicable to the cap 620.

A medical implant 700 configured to deliver a therapeutic substance is illustrated in FIG. 13. The medical implant 700 includes a fastener 710 and a cap 720. In the embodiment illustrated in FIG. 13, the fastener 710 is a break-off fastener. In some embodiments, the medical implant 700 may, in addition to the cap 720 in combination with a break-off fastener 710, include another component implant to which at least a portion of the break-off fastener couples. For example, as shown in FIG. 13, the cap 720 and fastener 710 are coupled to a cross-link device to be applied between two spinal rods in a spinal construct. The cross-link depicted is a CD HORIZON® X10 CROSSLINK® device provided by Medtronic, Inc., but any other device is contemplated within the scope of the claims. Other non-limiting examples of additional component implants include set screws of any variety, bone screws, pedicle screws, and locking screws.

The break-off fastener 710 illustrated on the right in FIG. 13 is a device that has not yet been operated and broken off. The fastener illustrated on the left is a fastener to which a torque has been applied so that a proximal portion 711 of the fastener 710 has been broken from a distal portion 712 of the fastener 710 after a predetermined amount of torque was applied to the fastener 710. A connection element may be configured to transfer a force which may be applied to manipulate at least a part of the fastener 710. In the illustrated embodiment, the opening in the distal portion 712 of the fastener 710, which the cap 720 is shown occupying, may serve as a connection element for the fastener 710.

The cap 720 is disposed at least in part in an internal opening in the break-off fastener 710. As illustrated in FIG. 13, at least a portion of the cap 720 remains in the medical implant 700, and in this embodiment at least a portion for the cap 720 remains in part of the break-off fastener 710, after the break-off fastener 710 is operated and broken off. The cap 720 of the illustrated embodiment includes a body that is a plug containing a therapeutic substance, and a linking mechanism configured to couple at least with the connection element of the fastener 710 to secure the cap 720 to the fastener 710. The body in the illustration of FIG. 13 is not distinguishable from the cap 720, and with reference to this embodiment, the cap 720 and body of the cap 720 will be referred to synonymously. In the illustrated embodiment, the linking mechanism includes external portions of the body of the cap 720. As illustrated with the distal portion 712 of the fastener 710, the cap 720 fits into an opening in the fastener 710. The cap 720 may include one or more of sizing, threads, fingers, and resilient material similar to the devices described in association with the insert 127 above to provide a secure fit between the cap 720 and the fastener 710.

In the illustrated embodiment, the cap 720 is configured to release at least a portion of the therapeutic substance when the cap 720 is exposed to an at least in part aqueous substance. The aqueous substances of some embodiments are bodily fluids. The materials from which the cap 720 and its component parts, including associated therapeutic substances, are made are essentially the same as the materials from which the cap 120 and its component parts are made, and the description above associated with the cap 120 is applicable to the cap 720.

Each of the caps 120, 220, 320, 420, 520, 620, and 720 is either placed during the implantation of existing implant systems or may be placed after components of existing implant systems have already been implanted. Therefore, the sequences for placing each of the listed caps is complementary to existing surgical procedures.

All patents and applications specifically list by number herein are hereby incorporated by reference herein in their entirety.

Terms such as proximal, distal, top, side, down and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A medical implant configured to deliver a therapeutic substance comprising:
a fastener with a connection element, wherein the connection element is configured to transfer a force which may be applied to manipulate at least a part of the fastener; and
a cap comprising:
a body that contains the therapeutic substance, and
a linking mechanism configured to couple at least with the connection element to secure the cap to the fastener;
wherein the body is configured to release at least a portion of the therapeutic substance when the body is exposed to an at least in part aqueous substance.

2. The medical implant of claim 1 wherein the fastener comprises a pedicle screw with a receiver member, and wherein the receiver member comprises a connection element that includes one or more openings.

3. The medical implant of claim 2 wherein the connection element comprises one or more pairs of openings on opposite, exterior sides of the receiver member.

4. The medical implant of claim 1 wherein the fastener comprises a pedicle screw construct that includes a set screw, and wherein the set screw comprises a connection element that includes one or more openings.

5. The medical implant of claim 1 wherein the fastener comprises a screw with a connection element comprising at least one opening in a proximal end of the screw.

6. The medical implant of claim 1 wherein the fastener comprises a screw with a proximal end that includes a connection element comprising threads on at least a portion of an exterior or the proximal end of the screw.

7. The medical implant of claim 1 wherein the fastener comprises a nut with a connection element comprising an exterior surface.

8. The medical implant of claim 1 wherein the cap captures at least a portion of the fastener between at least two opposing sides of the cap.

9. The medical implant of claim 1 wherein the body of the cap comprises a polymeric body configured to elute the therapeutic substance at a predetermined rate.

10. The medical implant of claim 1 wherein the body of the cap is at least in part porous and the therapeutic substance is at least in part disposed in pores of the body.

11. The medical implant of claim 1 wherein the linking mechanism of the cap comprises a protrusion configured to fit into an opening in the fastener.

12. The medical implant of claim 11 wherein the protrusion is generally round in cross-section and includes threads.

13. The medical implant of claim 1 wherein the linking mechanism of the cap comprises a hole configured to receive a portion of the fastener.

14. The medical implant of claim 1 wherein the linking mechanism of the cap comprises one or more resilient members configured to couple with the connection element of the fastener and provide resistance to separation of the cap from the fastener.

15. The medical implant of claim 1 wherein the cap is configured to wrap around a spinal rod that passes through a pedicle screw fastener, and wherein the linking mechanism of the cap at least couples with the spinal rod and the pedicle screw fastener by capturing at least the connection element of the fastener.

16. The medical implant of claim 1 wherein the cap is disposed at least in part in an internal opening in a break-off fastener, and wherein at least a portion of the cap is disposed in a part of the break-off fastener that remains in the medical implant after the break-off fastener is operated and broken off.

17. The medical implant of claim 1 wherein the therapeutic substance includes one or more of: antibiotic, antiseptic, analgesic, bone growth promoting substance, anti-inflammatant, anti-arrhythmic, anti-coagulant, antifungal agent, steroid, enzyme, immunosuppressant, antithrombogenic composition, vaccine, hormone, growth inhibitor, and growth stimulator.

18. The medical implant of claim 1, further comprising an adhesive applied between the cap and the fastener.

19. A cap configured to couple with a fastener and configured to deliver a therapeutic substance comprising:
a body that contains the therapeutic substance; and
a linking mechanism configured to couple at least with a connection element of the fastener to secure the cap to the fastener;
wherein the cap captures at least a portion of the fastener between at least two opposing sides of the cap; and
wherein the body is configured to release at least a portion of the therapeutic substance when the body is exposed to an at least in part aqueous substance.

20. The medical implant of claim 19 wherein the body of the cap comprises a polymeric body configured to elute the therapeutic substance at a predetermined rate.

21. The medical implant of claim 19 wherein the body of the cap is at least in part porous and the therapeutic substance is at least in part disposed in pores of the body.

22. The medical implant of claim 19 wherein the linking mechanism of the cap comprises a protrusion configured to fit into an opening in the fastener.

23. The medical implant of claim 19 wherein the linking mechanism of the cap comprises one or more resilient members configured to couple with the connection element of the fastener and provide resistance to separation of the cap from the fastener.

24. The medical implant of claim 19 wherein the cap is configured to wrap around a spinal rod that passes through a pedicle screw fastener, and wherein the linking mechanism of the cap at least couples with the spinal rod and the pedicle screw fastener by capturing at least the connection element of the fastener.

25. A cap in combination with a break-off fastener and configured to deliver a therapeutic substance comprising:
a body that contains the therapeutic substance; and
a linking mechanism coupled at least with the break-off fastener to secure the cap to the break-off fastener;
wherein the cap is disposed at least in part in an internal opening in the break-off fastener, and wherein at least a portion of the cap is disposed in a part of the break-off fastener that remains in the medical implant after the break-off fastener is operated and broken off; and
wherein the body is configured to release at least a portion of the therapeutic substance when the body is exposed to an at least in part aqueous substance.

26. The cap in combination with a break-off fastener of claim 25 further in combination with an implant to which at least a portion of the break-off fastener couples.
